(19) United States
(12) Patent Application Publication
(10) Pub. No.: US 2002/0133187 A1
(43) Pub. Date: Sep. 19, 2002

(54) Methods and apparatus for intercostal access

(76) Inventors: Robert F. Buckman, Radnor, PA (US); Rodney A. Brennaman, San Juan Capistrano, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/768,041

(22) Filed: Jan. 22, 2001

(51) Int. Cl. A61B 17/34

(52) U.S. Cl. 606/185

(57) ABSTRACT

The present invention provides improved devices and methods for establishing intercostal access for subsequent placement of minimally invasive direct cardiac massagers, chest tubes, defibrillation electrodes, and the like. In particular, the present invention provides devices and methods which facilitate rapid, safe, and sterile intercostal dissection for the subsequent deployment of minimally invasive direct cardiac massagers. An intercostal access punch device according to the present invention comprises a support having a proximal end and a distal end, a cutting tip attached to the distal end of the support, and means coupled to the support or cutting tip. The cutting tip is adapted to penetrate percutaneously through intercostal tissue between adjacent ribs to a thoracic cavity over a heart. Means coupled to the support or cutting tip penetrate tissue, engage at least one rib, and stop advancement of the cutting tip into the thoracic cavity.

### BACKGROUND OF THE INVENTION

1. Field of the Invention

The present invention relates generally to medical devices and methods. More particularly, the present invention relates to improved devices and methods for establishing intercostal access for subsequent placement of minimally invasive direct cardiac massagers, chest tubes, defibrillation electrodes, and the like.

Sudden cardiac arrest is a leading cause of death in most industrial societies. In order to resuscitate a victim of cardiac arrest, it is necessary to provide an adequate artificial circulation of blood to oxygenate the heart and brain by re-establishing the pumping function of the heart during the period between cardiac arrest and restoration of normal cardiac activity. Such a cardiac pumping function must be instituted at the earliest possible state. While in many cases it is theoretically possible to re-establish cardiac function, irreversible damage to vital organs, particularly the brain and the heart itself, will usually occur if sufficient blood flow is not re-established within a critical period of time from the moment of cardiac arrest. Such a period of time is measured ranging between four and six minutes.

A number of techniques have been developed to provide artificial circulation of blood to oxygenate the heart and brain during a cardiac arrest. Of particular interest to the present invention is the recent introduction of devices for performing minimally invasive direct cardiac massage. Such devices and methods are described in co-pending applications Nos. 09/087,665 filed May 29, 1998; 60/111,934 filed Dec. 11, 1998 (now abandoned); 09/344,440 filed Jun. 25, 1999; and 09/356,064 filed Jul. 19, 1999, assigned to the assignee of the present application. Generally, such methods rely on advancing a plurality of struts through an intercostal space to a region over a pericardium. The struts are opened along arcuate radially diverging paths between a posterior rib surface and the pericardium. The heart may then be pumped by directly engaging the opened struts against the pericardium to periodically compress the heart. Alternative minimally invasive direct cardiac massage devices and methods are also described in U.S. Pat. Nos. 5,582,580; 5,571,074; and 5,484,391 issued to Buckman, Jr. et al. and 5,931,850; 5,683,364; and 5,466,221 issued to Zadini et al., licensed to the assignee of the present application.

While direct cardiac massage approaches offer great promise, one issue to be resolved for the success and practical utility of direct cardiac massage devices is establishing safe, rapid, and sterile first entry into the chest cavity for such massagers. Previously proposed methods for gaining access into the chest cavity include sharp dissection with sharp surgical instruments or with a trocar-cannula assembly. However, such sharp dissection methods are disadvantageous as they often depend on blind advancement of a sharp instrument, which carries a risk of puncturing and/or lacerating the heart, coronary vessels, or the surrounding structures. Other proposed methods employ a combination of sharp and blunt dissection to establish intercostal access. These methods typically employ a sharp surgical instrument, such as a surgical knife, lancet, scalpel, blade, and the like, to make a partial incision through the skin overlying the intercostal space, and then advancing a blunt member through the intercostal space above the pericardium. Likewise, these methods also suffer drawbacks as such multi-step procedures are often time-consuming, slow, and conflict with the need for a rapid institution of cardiopulmonary resuscitation. Furthermore, such multi-step approaches still require a certain level of surgical skill to avoid any potential injuries to intrathoracic organs. Such methods in general also require the use of surgical gloves, which in turn adds to the set up time for a resuscitation procedure where the need for performing urgently is critical. Moreover, these access protocols are not inherently antiseptic, which is an important safeguard as most cardiac arrest occur outside of a hospital setting. Thus, none of the prior art methods or devices have been entirely satisfactory.

For these reasons, it would be desirable to provide improved devices and methods for establishing intercostal access for subsequent placement of minimally invasive direct cardiac massagers, chest tubes, defibrillation electrodes, and the like. In particular, it would be desirable to provide an apparatus and methods which facilitate rapid, safe, and sterile first entry into the chest cavity for the subsequent deployment of minimally invasive direct cardiac massagers. It would be further desirable if such devices minimize the level of surgical skill required for implementation and eliminate the need for surgical gloves. The devices and methods should allow for sharp swift access without injury to intrathoracic organs while maintaining an antiseptic environment. At least some of these objectives will be met by the devices and methods of the present invention described hereinafter.

2. Description of the Background Art

Devices and methods for minimally invasive direct cardiac massage through intercostal dissection are described co-pending applications Nos. 09/087,665 filed May 29, 1998; 60/111,934 filed Dec. 11, 1998 (now abandoned); 09/344,440 filed Jun. 25, 1999; and 09/356,064 filed Jul. 19, 1999. Devices and methods for minimally invasive direct cardiac massage through blunt first entry methods are described by Zadini et al. in U.S. Pat. Nos. 5,931,850; 5,683,364; and 5,466,221, licensed to the assignee of the present application. Devices and methods for minimally invasive direct cardiac massage through sharp intercostal dissection methods are described by Buckman, Jr. et al. in U.S. Pat. Nos. 5,582,580; 5,571,074; and 5,484,391. U.S. Pat. No. 3,496,932, issued to Prisk describes a trocar-cannula assembly for introducing a cardiac massage device to a space between the sternum and the heart. Dissectors employing inflatable components are described in U.S. Pat. Nos. 5,730,756; 5,730,748; 5,716,325; 5,707,390; 5,702,417; 5,702,416; 5,694,951; 5,690,668; 5,685,826; 5,667,520; 5,667,479; 5,653,726; 5,624,381; 5,618,287; 5,607,443; 5,601,590; 5,601,589; 5,601,581; 5,593,418; 5,573,517; 5,540,711; 5,514,153; and 5,496,345.

The full disclosures of each of the above references are incorporated herein by reference.

### SUMMARY OF THE INVENTION

The present invention provides improved devices and methods for establishing intercostal access for subsequent placement of minimally invasive direct cardiac massagers, chest tubes, defibrillation electrodes, and the like. In particular, the present invention provides devices and methods which facilitate rapid, safe, and sterile intercostal dissection for the subsequent deployment of minimally invasive direct cardiac massagers. Moreover, the present invention minimizes the level of surgical skill required for implementation of the present device and eliminates the need for surgical gloves.

In a first aspect of the present invention, an intercostal access punch device according to the present invention comprises a support having a proximal end and a distal end, a cutting tip attached to the distal end of the support, and means coupled to the support or cutting tip. The cutting tip, which may comprise a blade or trocar, is adapted to penetrate percutaneously through intercostal tissue between adjacent ribs to a thoracic cavity over a heart. Means coupled to the support or cutting tip penetrate tissue, engage at least one rib, and stop advancement of a leading tip of the cutting tip into the thoracic cavity.

The present access punch device provides many significant advantages. For example, the access punch utilizes the ribs as a reference point so that means coupled to the support or cutting tip penetrate tissue and engage at least one rib to stop advancement of the cutting tip into the thoracic cavity, regardless of individual variability in the anterior surface of the rib and/or the depth of the thoracic cavity. As such, rapid sharp dissection with the cutting tip can be safely implemented without fear of blind advancement and/or accidentally puncturing intrathoracic organs. Moreover, a safe and rapid access device improves the effectiveness and usefulness of subsequent direct cardiac massage, where the need for performing urgently and safely is critical. Additionally, implementation of the present access punch device minimizes the need for specialized surgical skill and, consequently, the device of the present invention may be applied by semi-skilled persons, such as paramedic personnel and the like. This will ultimately effect the rapidity of dissection, and hence, the usefulness of the device.

The tissue penetrating means to stop advancement of a leading tip of the cutting tip can take a variety of forms. Conveniently, it can be a shoulder formed integrally with the cutting tip in a single blade structure. The blade will penetrate tissue across a width larger than an intercostal space. The shoulder(s) will engage anterior surface(s) of rib(s) when the leading tip of the cutting tip has reached the thoracic cavity (typically being within ∀5 mm of the cavity, usually within ∀3 mm of the cavity). Alternatively, the tissue penetrating means can be a pin, blade, electrosurgical tip, or other tissue penetrating structure which is formed separately from the cutting tip. Such separate structure will be mechanically coupled to the tip, however, so that the structure will penetrate tissue and eventually engage an anterior rib surface to halt advancement of the cutting tip at the appropriate point. Optionally, a separate tissue penetrating structure can be positionally adjustable relative to the tissue penetrating tip to account for patients having different body sizes.

The access punch device may utilize various cutting tip configurations, typically comprising one to three blades or trocars. In a preferred embodiment the access punch comprises a single blade. In an alternate embodiment, the access punch comprises two blades perpendicular to each other to form a cross shape. The cutting tip may have a cutting edge that is triangular, serrated, curved, or a combination thereof. In particular, the cutting tip will usually form a sharp leading distal tip (i.e. triangular or arrow pointed tip). The leading distal tip will typically have a maximum length of 10 mm or less, preferably a maximum length of 5 mm or less, from the stopping means or shoulder of the cutting tip. The cutting tip will typically have a minimum included diameter of 5 mm or greater, preferably a minimum diameter of 10 mm or greater.

The access punch device of the present invention may further include a recessed housing structure attached to the support for housing the cutting tip before intercostal penetration. Preferably, the housing structure will have a generally bell shape. The recessed housing structure advantageously provides a sterile enclosure for the cutting tip prior to use and acts as a protective cover for the user and during storage. A resilient spring may also be disposed on the support and rest against the housing structure. The spring will automatically retract the cutting tip back into the housing structure following intercostal penetration. Additionally, the access punch device may further comprise a moveable handle at the proximal end of the support.

To promote sterility of the presently claimed access punch, a bottom side of the recessed housing structure may be enclosed by having a first penetrable cover or membrane disposed over at least a bottom side of the housing structure, and a second cover disposed over the first cover. The second cover may additionally have a pull-off loop or tab attached to it. It will be appreciated that the covers may be separate components or formed integrally with the housing structure. The first and second covers may optionally be disposed over part of the device or over the entire structure of the device. Suitable materials for the membranes include latex, polyethylene, polypropylene, polyester, and the like. The access punch device may further comprise an outer pouch or bag which encloses at least a bottom portion of the housing structure. An important advantage of such dual covers and/or an outer pouch is that such structures provide and maintain a sterile environment before and during use of the access device. This is particularly beneficial as most cardiac arrest occur outside a hospital setting in the field. Furthermore, the sterile covers and/or outer pouch eliminate the need for additional packaging and/or surgical gloves, which in turn reduces the set-up time of such procedures and increases the rapidity of device implementation.

The access punch device may additionally comprise a detachable adhesive skin contacting surface which may be a part of the first cover or the housing structure. The adhesive skin contacting surface will typically form a patch around the dissected tissue to maintain near normal inter-thoracic pressure. As another alternative, the housing structure of the access punch device may have a bottom portion which is detachable from a top portion of the housing structure to form an access port over the dissected tissue. An access port allows for subsequent entry of devices such as minimally invasive direct cardiac massagers, chest tubes, defibrillator electrodes, and the like.

In a second aspect of the present invention, an intercostal access punch comprises a handle and a cutting tip attached to the handle. The cutting tip may have a cutting edge with a profile having a leading tip and at least one shoulder. The leading tip is sized to advance between adjacent ribs and the shoulder is positioned to engage at least one rib and stop advancement of the leading tip prior to entry of the leading tip into a thoracic cavity as the cutting tip is percutaneously advanced toward a heart and internal organs.

In a third aspect of the present invention, methods for establishing percutaneous intercostal access are provided. One method comprises advancing a cutting tip having a tissue penetrating stop element coupled to the cutting tip through intercostal tissue. The stop element engages at least one rib after the cutting tip dissects tissue between adjacent ribs but before the cutting tip enters a thoracic cavity.

The advancing step is typically carried out by pressing a housing structure against a skin surface and depressing a moveable handle attached to the cutting tip so that the cutting tip cuts through the skin surface to a preselected tissue depth external to the thoracic cavity. Typically, the amount of force required to depress the handle will be in a range from 0.5 lbs to 3 lbs. Cutting tip penetration may be limited to a preselected tissue depth in a range typically from 0.5 cm to 5 cm, preferably in a range from 0.5 cm to 1 cm, below an outer rib surface. The preselected tissue depth will usually comprise an inner rib surface, or may alternatively comprise a thoracic pleural lining. Preferably, the advanced cutting tip is automatically retracted back into the housing structure via a resilient spring. The resilient spring will typically require 0.25 lbs to 1 lbs of force to be fully compressed.

Preferably, an outer pouch disposed over at least a bottom portion of the housing structure may be removed prior to advancing the cutting tip. An outer cover disposed on a bottom side of the housing structure may also be removed via a pull-off loop or tab prior to cutting tip advancement. The cutting tip may also cut through an inner cover enclosing a bottom side of the housing structure prior to cutting through the skin surface to the preselected tissue depth.

Following cutting tip advancement, an adhesive skin contacting surface of the inner cover or housing structure may be detached on the skin surface so as to form an access patch around the dissected tissue. Optionally, a bottom portion of the housing structure may be detached from a top portion of the housing structure so as to form an access port over the dissected tissue. Still further optionally, a direct cardiac massage device may be advanced following intercostal dissection.

### BRIEF DESCRIPTION OF THE DRAWINGS

FIG. 1 is a perspective view of an exemplary access punch device for establishing intercostal access constructed in accordance with the principles of the present invention.

FIG. 2 is a bottom view of the device of FIG. 1.

FIG. 3 illustrates an alternate embodiment of the device of the present invention utilizing two blades.

FIG. 4 is a bottom view of the device of FIG. 3.

FIGS. 5A-5C illustrate alternative cutting tip configurations which may be employed in the device of FIGS. 1 or 3.

FIGS. 6A-6B illustrate a detachable adhesive skin contacting surface which may be employed in the device of FIGS. 1 or 3.

FIGS. 7A-7B illustrate a detachable housing structure which may be employed in the device of FIGS. 1 or 3.

FIG. 8 is a cross-sectional view illustrating the heart underneath a patient's rib cage.

FIGS. 9 and 10 illustrate a method according to the present invention employing the access punch device of FIG. 1.

### DESCRIPTION OF THE SPECIFIC EMBODIMENTS

The present invention provides improved devices and methods for establishing intercostal access for subsequent placement of minimally invasive direct cardiac massagers, chest tubes, defibrillator electrodes, and the like. In particular, the present invention provides devices and methods which facilitate rapid, safe, and sterile intercostal dissection for the subsequent deployment of minimally invasive direct cardiac massagers.

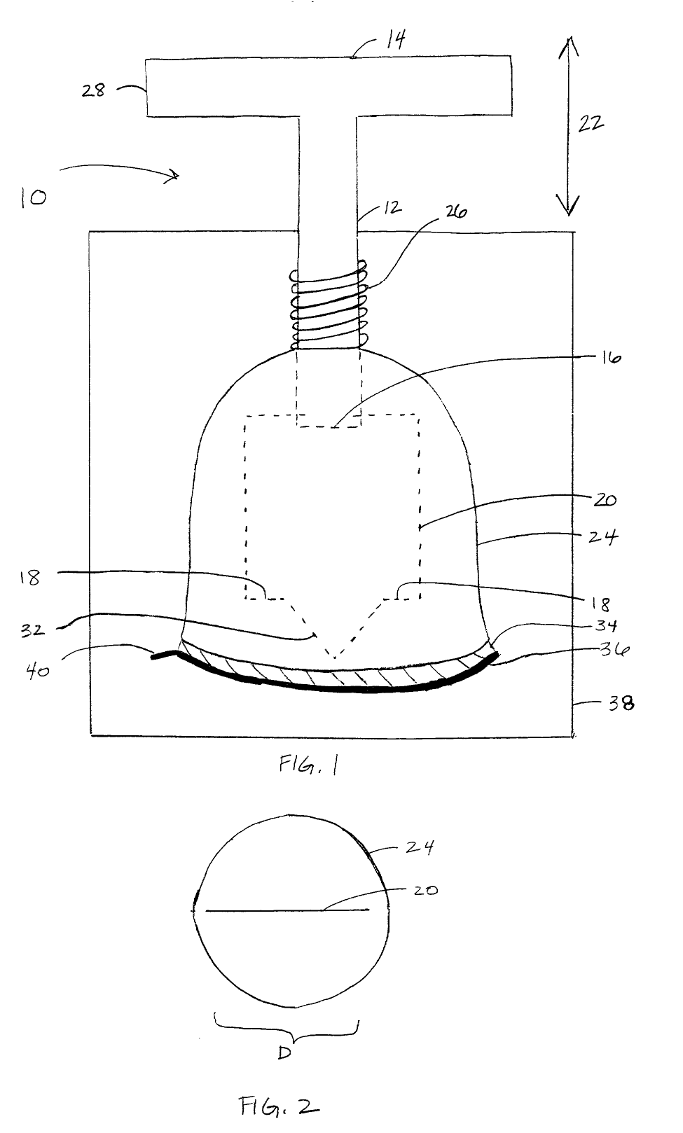

Referring now to FIGS. 1 and 2, an exemplary access punch device 10 constructed in accordance with the principles of the present invention for establishing intercostal access is illustrated. The access punch device 10 comprises a support or shaft 12 having a proximal end 14 and a distal end 16, a cutting tip 20 attached to the distal end 16 of the support 12, and means 18 coupled to the cutting tip 20, as shown in FIG. 1, or alternatively to the support 12. The cutting tip 20, which may comprise a blade or trocar, is adapted to penetrate percutaneously through intercostal tissue between adjacent ribs to a thoracic cavity over a heart. Means 18 coupled to the cutting tip 20 or the support 12 penetrate tissue, engage at least one rib, and stop advancement of the cutting tip 20 into the thoracic cavity. As discussed above, the access punch device 10 utilizes the ribs as a reference point so that means 18 coupled to the cutting tip 20 or the support 12 penetrate tissue and engage at least one rib to stop advancement of the cutting tip 20 above the thoracic cavity, regardless of individual variability in the anterior surface of the rib and/or the depth of the thoracic cavity. As such, rapid sharp dissection with the cutting tip 20 and the means 18 coupled to the cutting tip 20 or the support 12 can be safely implemented without fear of blind advancement and/or accidentally puncturing intrathoracic organs. Additionally, implementation of the present access punch device 10 minimizes the need for specialized surgical skill. It will be appreciated that the following depictions are for illustration purposes only and does not necessarily reflect the actual shape, size, or dimensions of the access device 10. This applies to all depictions hereinafter.

The tissue penetrating means 18 to stop advancement of a leading tip 32 of the cutting tip 20 can take a variety of forms. The tissue penetrating means 18 may comprise a lateral extension of the cutting tip 20 which is configured to engage an anterior surface of a rib when a leading tip 32 of the cutting tip 20 reaches the thoracic cavity. Alternatively, the tissue penetrating means 18 can comprise a tissue-penetrating pin, blade, electrosurgical tip, or other tissue penetrating structure which is configured to penetrate tissue when the cutting tip 20 is advanced and to engage an anterior surface of a rib when a leading tip 32 of the cutting tip 20 reaches the thoracic cavity. Such separate structure will be mechanically coupled to the cutting tip 20, however, so that the structure will penetrate tissue and eventually engage an anterior rib surface to halt advancement of the cutting tip 20 at the appropriate point.

The access punch device 10 of the present invention may further include a recessed housing structure 24 attached to the support 12 for housing the cutting tip 20 before intercostal penetration, as illustrated in FIG. 2. Preferably, the housing structure 24 will have a generally bell shape and be made of plastic, metal, rubber, or like materials. The recessed housing structure 24 advantageously provides a sterile enclosure for the cutting tip 20 prior to use and acts as a protective cover for the user and during storage. A resilient spring 26 may also disposed on the support 12 and rest against the housing structure 24. The spring 26 will automatically retract the cutting tip 20 back into the housing structure 24 following intercostal penetration. Additionally, the access punch device 10 may further comprise a moveable handle 28 at the proximal end 14 of the shaft 12, which moves in an axial direction, as depicted by arrow 22. The handle 28 and support 12 will usually be integrally formed, as illustrated in FIG. 1, and will be made of plastic, metal, rubber, or like materials.

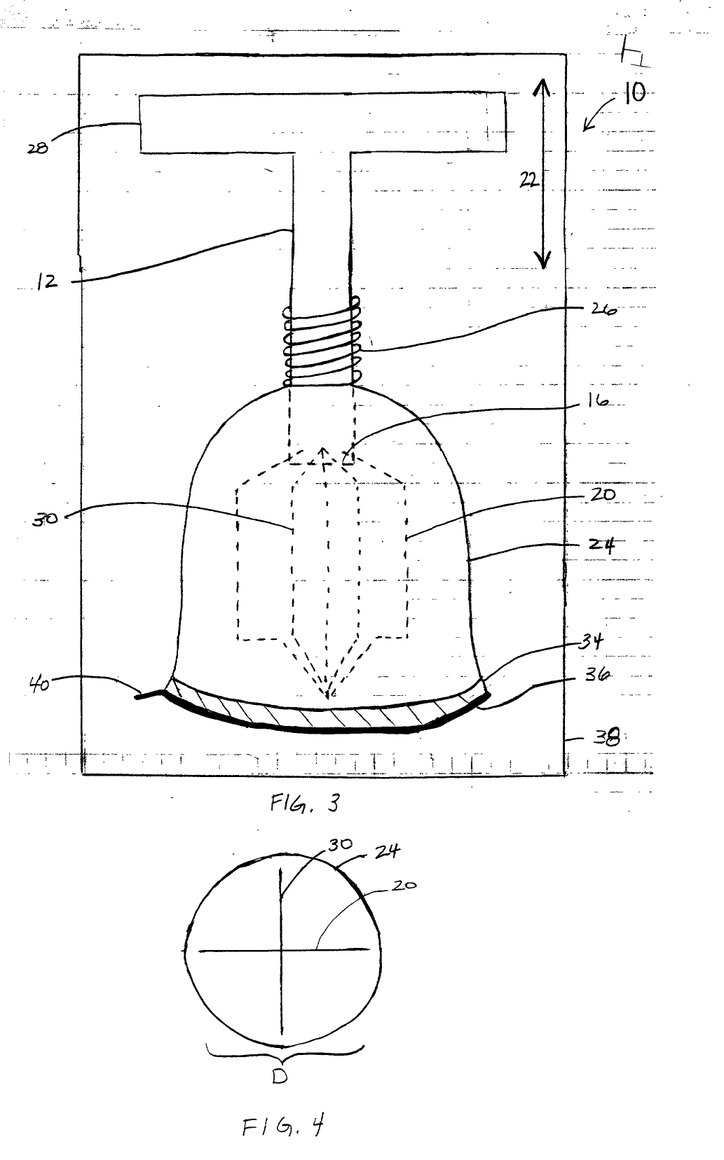

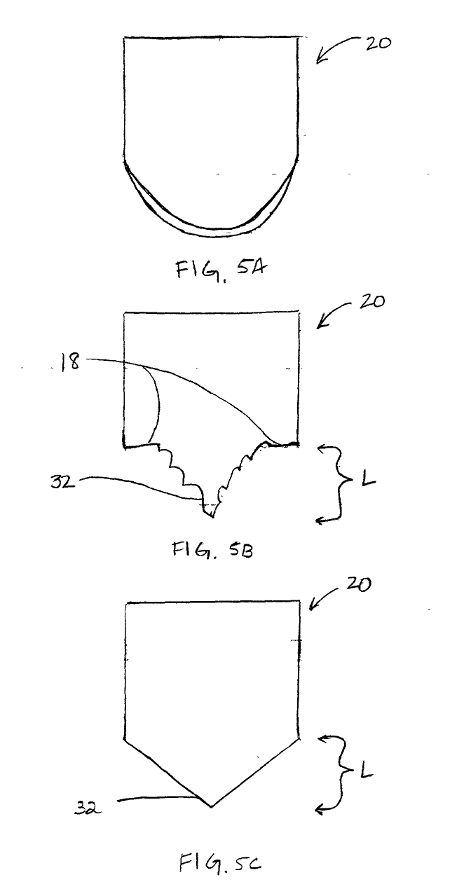

With reference to FIGS. 3 and 4, an alternative cutting tip configuration for access punch 10 comprises two blades 20 and 30. The two blades 20 and 30 will typically be perpendicular to each other to form a cross shape as shown in FIG. 4. The cutting tip 20 will typically have a minimum included diameter D of 5 mm or greater, preferably a minimum diameter D of 1 cm or greater, so that means 18 coupled to cutting tip 20 engage at least one rib.

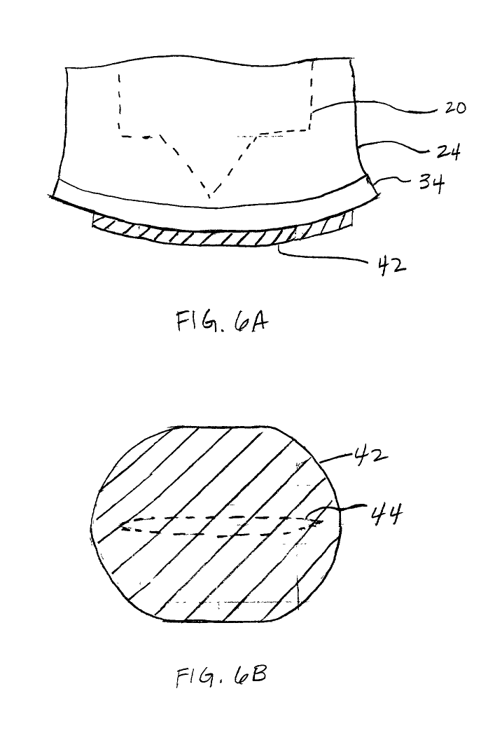

Referring now to FIGS. 5A-5C, cutting tip 20 may comprise a blade having cutting edges that are curved (FIG. 5A), serrated (FIG. 5B), triangular (FIGS. 5B and 5C), or a combination thereof. In particular, the at least one cutting tip 20 will usually form a leading distal tip 32 (i.e. sharp triangular or arrow pointed tip). The triangular distal tip 32 will typically have a maximum length L of 10 mm or less, preferably a maximum length L of 5 mm or less, from the stopping means or shoulder 18 of the cutting tip 20 to further ensure that the cutting tip tip 32 does not accidentally puncture the heart or lungs.

To promote sterility of the presently claimed access punch 10, a bottom side of the recessed housing structure 24 may be enclosed by having a first penetrable cover or membrane 34 disposed over at least a bottom side of the housing structure 24, and a second cover 36 disposed over the first cover 34. The second cover 36 may additionally have a pull-off loop 40 or tab attached to it. It will be appreciated that the covers 34, 36 may be separate components or formed integrally with the housing structure 24. Suitable materials for the membranes include latex, polyethylene, polypropylene, polyester, and the like. The access punch device 10 may further comprise an outer pouch or bag 38 enclosing at least a bottom portion of the recessed housing structure 34, as shown in FIG. 1, or over the entire structure of the device 10, as depicted in FIG. 3. As described above, an important advantage of dual covers 34, 36 and/or an outer pouch 38 is that such membranes eliminate the need for surgical gloves while providing and maintaining a sterile environment before and during use of the access device 10.

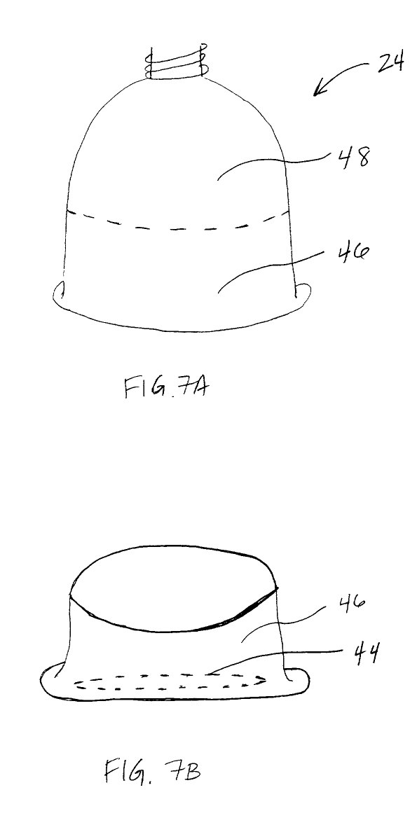

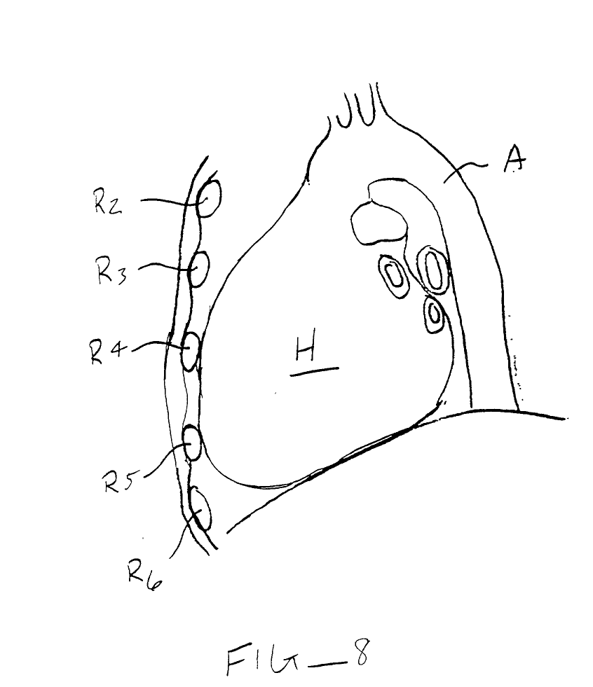

Referring now to FIGS. 6A and 6B, the access punch device 10 is illustrated with a detachable adhesive skin contacting surface 42. The skin contacting surface 42 may be a part of the first cover 34, as illustrated, or a part of the housing structure 24. The adhesive skin contacting surface 42 will typically form a patch around the dissected tissue 44 to maintain near normal inter-thoracic pressure. The adhesive skin contacting surface 42 will be made of suitable materials, such as, hydrogel, adhesive coated paper, rubber, plastic, and the like.

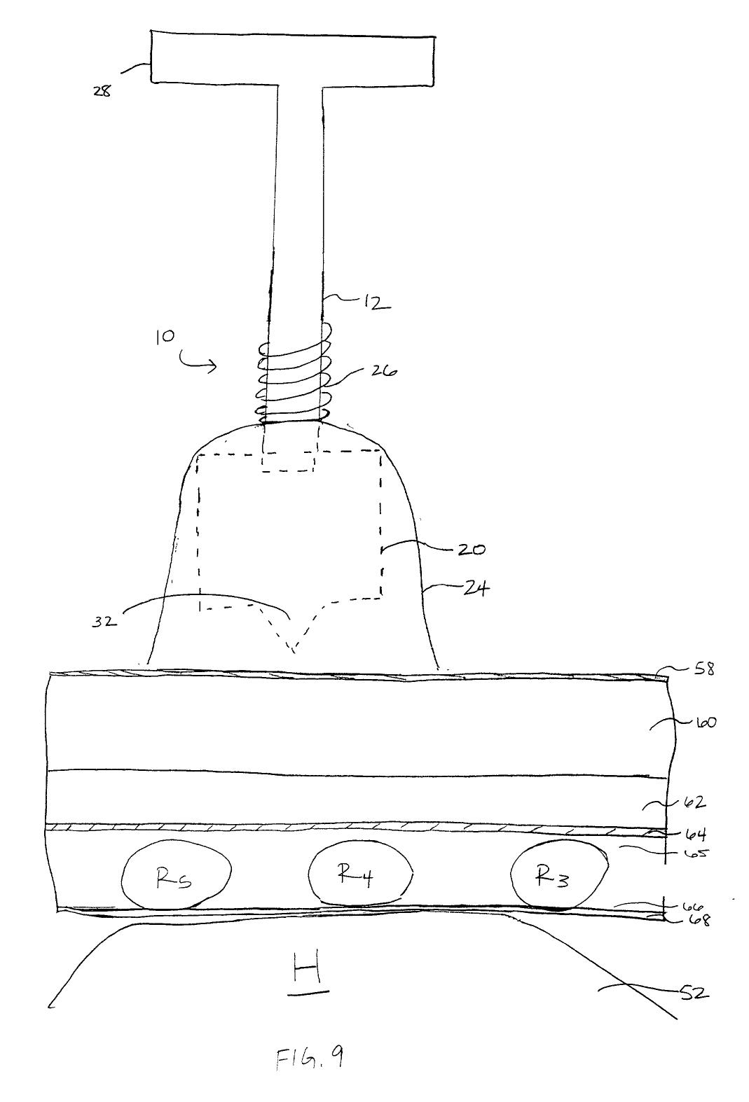

Referring now to FIGS. 7A and 7B, the housing structure 24 of the access punch device 10 may alternatively have a bottom portion 46 which is detachable from a top portion 48 of the housing structure 24 to form an access port 46 over the dissected tissue 44. An access port 46 allows for subsequent entry of devices such as minimally invasive direct cardiac massagers, chest tubes, defibrillator electrodes, and the like.

Referring now to FIG. 8, a patient's heart H is shown in a cross-section between ribs R, where n indicates the rib number. The aorta A is also shown extending from the top of the heart.

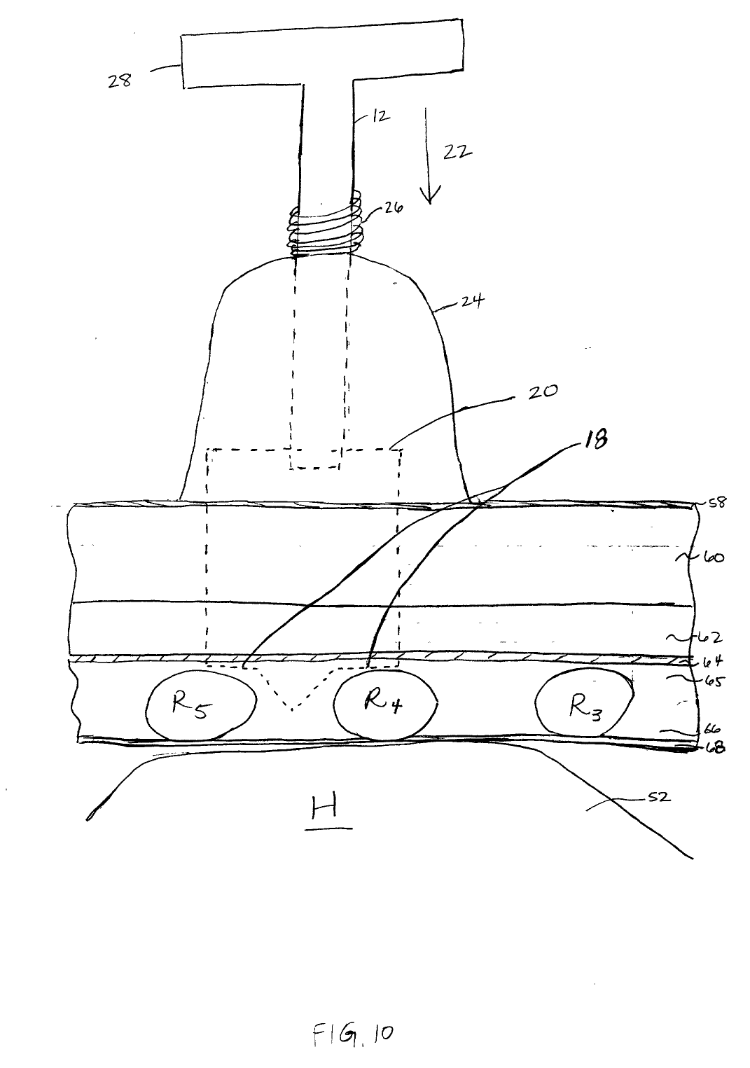

Referring now to FIGS. 9 and 10, a first exemplary method for establishing percutaneous intercostal access with the access punch device 10 of FIG. 1 will be described. A cutting tip 20 having a tissue penetrating stop element 18 coupled to the cutting tip 20 is advanced through intercostal tissue. The stop element 18 engages at least one rib after the cutting tip 20 dissects tissue between adjacent ribs but before the cutting tip 20 enters a thoracic cavity 52. Typically, the stop element 18 of the cutting tip 20 will rest against rib R₄ or R₅.

The advancing step is typically carried out by pressing a housing structure 24 against a skin surface 58 and depressing a moveable handle 28 attached to the cutting tip 20 so that the cutting tip 20 cuts through the skin surface 58 to a preselected tissue depth external to the thoracic cavity 52. As illustrated in FIG. 10, a subcutaneous fat layer 60, a muscle layer 62, and a thin facia layer 64 lie respectively beneath the skin surface 58. Cutting tip penetration may be limited to a preselected tissue depth in a range typically from 0.5 cm to 5 cm, preferably in a range from 0.5 cm to 1 cm, below an outer rib surface 65. As noted earlier, the exact depth of penetration depends ultimately on the specific patient as there is individual variability in the depth of the thoracic cavity. The preselected tissue depth will usually comprise an inner rib surface 66, as shown, or may alternatively comprise a thoracic pleural lining 68. Typically, the amount of force required to depress the handle 28 in direction 22 will be in a range from 0.5 lbs. to 3 lbs. Preferably, the advanced cutting tip 20 is automatically retracted back into the housing structure 24 via a resilient spring 26 (FIG. 9). The resilient spring 26 will typically require 0.25 lbs. to 1 lbs. of force to be fully compressed.

Preferably, an outer pouch 38 disposed over at least a bottom portion of the housing structure 24 will be removed prior to advancing the cutting tip 20. An outer cover 36 disposed on a bottom side of the housing structure 24 may also be removed via a pull-off loop 40 or tab prior to advancing the cutting tip 20. The cutting tip 20 may also cut through an inner cover 34 enclosing the bottom side of the housing structure 24 prior to cutting through the skin surface 58 to the preselected tissue depth.

Following cutting tip advancement, an adhesive skin contacting surface 42 of the inner cover 34 or the housing structure 24 may be detached on the skin surface 58 so as to form an access patch around the dissected tissue 44. Optionally, a bottom portion 46 of the housing structure 24 may be detached from a top portion 48 of the housing structure 24 so as to form an access port over the dissected tissue 44. Still further optionally, a direct cardiac massage device may be advanced following intercostal dissection.

Although certain preferred embodiments and methods have been disclosed herein, it will be apparent from the foregoing disclosure to those skilled in the art that variations and modifications of such embodiments and methods may be made without departing from the true spirit and scope of the invention. Therefore, the above description should not be taken as limiting the scope of the invention which is defined by the appended claims.

What is claimed is:

1. An intercostal access device comprising:
a support having a proximal end and a distal end; and
a cutting tip attached to the distal end of the support, said cutting tip being adapted to penetrate percutaneously through intercostal tissue between adjacent ribs to a thoracic cavity over a heart; and
means coupled to the support or cutting tip for penetrating tissue, engaging at least one rib, and stopping advancement of the cutting tip into the thoracic cavity.

2. An access device as in claim 1, wherein the tissue penetrating means comprises a lateral extension of the cutting tip which is configured to engage an anterior surface of a rib when a leading tip of the cutting tip reaches the thoracic cavity.

3. An access device as in claim 1, wherein the tissue penetrating means comprises a separate tissue-penetrating pin which is configured to penetrate tissue when the cutting tip is advanced and to engage an anterior surface of a rib when a leading tip of the cutting tip reaches the thoracic cavity.

4. An access device as in claim 1, further comprising a recessed housing structure attached to the support for housing the cutting tip before intercostal penetration.

5. An access device as in claim 4, further comprising a resilient spring disposed on the support and resting against the housing structure, the spring automatically retracting the cutting tip back into the housing structure following intercostal penetration.

6. An access device as in claim 4, wherein the housing structure has a generally bell shape.

7. An access device as in claim 4, wherein a bottom side of the housing structure is enclosed by at least one cover.

8. An access device as in claim 7, further comprising a pouch enclosing at least a bottom portion of the housing structure.

9. An access device as in claim 7, wherein the at least one cover has a detachable adhesive skin contacting surface which forms an access patch around the dissected tissue.

10. An access device as in claim 4, wherein a bottom portion of the housing structure is detachable from a top portion of the housing structure to form an access port over the dissected tissue.

11. An access device as in claim 1, wherein the cutting tip is a triangular, serrated, or curved blade.

12. An access device as in claim 1, wherein the cutting tip forms a triangular point.

13. An access device as in claim 15, wherein the triangular point has a length less than 5 mm.

14. An access device as in claim 1, wherein the cutting tip has a diameter greater than 1 cm.

15. An access device as in claim 1, further comprising a moveable handle at the proximal end of the support.

16. An intercostal access device comprising:
a handle; and
a cutting tip attached to the handle, wherein the cutting tip has a cutting edge with a profile having a leading tip and at least one shoulder, wherein the leading tip is sized to advance between adjacent ribs and the shoulder is positioned to engage at least one rib and stop advancement of the leading tip prior to entry of the leading tip into a thoracic cavity as the cutting tip is percutaneously advanced toward a heart.

17. A method for percutaneous intercostal access, said method comprising:
advancing a cutting tip having a tissue penetrating stop element coupled to the cutting tip through intercostal tissue, wherein the stop element engages at least one rib after the cutting tip dissects tissue between adjacent ribs but before the cutting tip enters a thoracic cavity.

18. A method as in claim 17, further comprising automatically retracting the advanced cutting tip back into a housing structure with a resilient spring.

19. A method as in claim 17, wherein the advancing step is carried out by pressing a housing structure against a skin surface and depressing a moveable handle attached to the cutting tip so that the cutting tip cuts through the skin surface to a preselected tissue depth external to the thoracic cavity.

20. A method as in claim 19, further comprising removing an outer pouch disposed over at least a bottom portion of the housing structure prior to cutting tip advancement.

21. A method as in claim 20, further comprising removing an outer cover disposed on a bottom side of the housing structure prior to cutting tip advancement.

22. A method as in claim 21, wherein the cutting tip cuts through an inner cover enclosing the bottom side of the housing structure prior to cutting through the skin surface to the preselected tissue depth.

23. A method as in claim 22, farther comprising detaching an adhesive skin contacting surface of the inner cover on the skin surface so as to form an access patch around the dissected tissue.

24. A method as in claim 19, further comprising detaching a bottom portion of the housing structure from a top portion of the housing structure so as to form an access port over the dissected tissue.

25. A method as in claim 19, wherein the preselected tissue depth comprises an inner rib surface.

26. A method as in claim 19, where in the preselected tissue depth comprises a thoracic pleural lining.

27. A method as in claim 19, wherein the preselected tissue depth is in a range from 0.5 cm to 5 cm.

28. A method as in claim 17, wherein the cutting tip is a triangular, serrated, or curved blade.

29. A method as in claim 17, wherein the cutting tip forms a leading tip.

30. A method as in claim 29, wherein the leading tip has a length less than 5 mm.

31. A method as in claim 17, wherein the cutting tip has a diameter greater than 1 cm.

32. A method as in claim 17, further comprising advancing a direct cardiac massage device following intercostal advancement.
